(19) United States
(12) Patent Application Publication
(10) Pub. No.: US 2023/0310374 A1
(43) Pub. Date: Oct. 5, 2023

(54) COMPOSITIONS AND METHODS FOR TREATING HEADACHE OR FACIAL PAIN

(71) Applicant: ATAI Life Sciences AG, Berlin (DE)

(72) Inventors: Srinivas G. RAO, Encinitas, CA (US); Glenn SHORT, Scituate, MA (US); Carrie BOWEN, Uxbridge, MA (US)

(21) Appl. No.: 18/192,603

(22) Filed: Mar. 29, 2023

Related U.S. Application Data
(63) Continuation-in-part of application No. 17/957,851, filed on Sep. 30, 2022.
(60) Provisional application No. 63/250,595, filed on Sep. 30, 2021.

(51) Int. Cl. A61K 31/4045; A61P 25/06

(52) U.S. Cl. CPC A61K 31/4045; A61P 25/06

(57) ABSTRACT

The present disclosure provides new methods of treating headache or facial pain with 5-methoxytryptamine or 5-hydroxytryptamine and compositions thereof.

### CROSS-REFERENCE TO RELATED APPLICATION

The present application is a continuation-in-part of U.S. patent application Ser. No. 17/957,851, filed Sep. 30, 2022, which claims the benefit of priority to U.S. Provisional Application No. 63/250,595, filed Sep. 30, 2021, the contents of which are hereby incorporated by reference in its entirety.

### BACKGROUND OF THE INVENTION

### (i) Field of the Invention

The present invention relates to compositions and methods for treating headache or facial pain and, in some embodiments, to treatment of headaches in a patient using certain tryptamine compounds as well as prodrugs or salts thereof.

### (ii) Description of Related Art

Migraine and cluster headache are episodic and often chronic headache disorders that can lead to significant disability (Andersson et al., 2017; Harriott et al., 2019). Migraine has a worldwide prevalence of nearly 15% and is associated with severe recurrent headaches with accompanying sensitivity to light and/or sound, nausea and vomiting. The condition is heterogeneous and involves both genetic and environmental factors. Migraine pain is mediated by activation of trigeminal nociceptors innervating meningeal tissues, including dural arteries and sinuses, and release of neuropeptides, such as calcitonin gene related peptide (CGRP), substance P, neurokinin A and/or pituitary adenylate cyclase activating peptide (PACAP). Additional brain areas, such as the cortex, thalamus and hypothalamus, also are believed to be involved in the generation of a migraine episode (Puledda et al., 2017). Cluster headache is a rarer, sometimes treatment-resistant and particularly painful condition with a prevalence around 1 in 1000 individuals and without any pharmacological treatments specifically developed for this disorder. There is a need in the art for new methods of treating headaches.

Acute pharmacological treatment of migraine may include ergotamine, triptans, CGRP receptor antagonists (Gepants) or 5-HT1F receptor agonists (ditans) (Puledda et al., 2017). Ergotamine is a non-hallucinogenic ergot alkaloid originally isolated from fungus with anti-migraine activity that is believed to be mediated by agonism at serotonin 5-HT1B/1D receptors, but its lack of pharmacological selectivity leads to unwanted side effects (Yu, 2008). Triptans, such as sumatriptan, are selective serotonin 5-HT1B/1D receptor agonists that modify release of neurotransmitters from the central terminals of trigeminal primary afferent fibers, are clinically useful migraine-abortive drugs with improved tolerability over ergotamine, and also are prescribed for the treatment of cluster headache. However, side effects (e.g., cardiovascular) limit the use of triptans, and lack of efficacy or recurrence of migraine symptoms has been seen in over 50% of cases (Puledda et al., 2017). Gepants, such as ubrogepant which was approved in 2019, are effective acute migraine treatments with improved tolerability over triptans, but some compounds in this class have been associated with liver toxicity (Puledda et al., 2017). Similarly, the ditans, such as lasmiditan which was approved by the FDA in 2019, exhibit acute anti-migraine activity without cardiovascular effects, although mild central nervous system side effects have been reported (Puledda et al., 2017).

Lack of efficacy and adverse effects of available medications have prompted the search for alternative treatments for acute headache disorders. A few studies and anecdotal reports indicate that psychoactive tryptamines, a structural class of compounds containing an indolealkylamine backbone, are effective against migraine and cluster headache, often at sub-psychoactive doses and with long-lasting effects. Andersson et al. (2017) reported the following tryptamines as being specifically discussed in online forums for the alleviation of headache disorders: psilocybin, LSD, 1P-LSD, AL-LAD, D-Lysergic acid amide, DMT, 4-AcO-MET, 4-AcO-DMT, 4-HO-MiPT, 4-HO-MET, 5-MeO-MiPT and 5-MeO-DALT. The non-psychoactive LSD analog, 2-bromo-LSD or BOL-148, also has shown a promising efficacy versus side effect profile for the treatment of cluster headache. Madsen et al. 2022 reports that three doses of psilocybin, each separated by seven days, prophylactically reduced chronic cluster headache attack frequency in a 10-patient open-label study (Madsen et al., 2022). One of these 10 patients experienced complete remission for 21 weeks, beginning one day after the first psilocybin session. These indole alkaloid compounds interact with serotonin receptors, including 5-HT2A, 5-HT1B and/or 5-HT1D, and have structural similarity to the triptans.

Dimethyltryptamine (DMT) and its analogs belong to a class of drugs referred to as psychedelics (“mind-manifesting” drugs). Specifically, DMT and analogs are considered 5-hydroxytryptaminergic (serotonergic) psychedelics, like other tryptamines such as psilocybin, ergolines such as lysergic acid diethylamide (LSD). DMT is a naturally occurring alkaloid found in animal and plant species. Like DMT, the analogs 5-methoxydimethyltryptamine (5-MeO-DMT) and 5-hydroxydimethyltryptamine (5-OH-DMT, bufotenine) are naturally occurring alkaloids found in some toad and plant species. There remains a need in the art for improved compositions and methods for treating patients using DMT and its analogs.

### SUMMARY OF THE DISCLOSURE

In one aspect, the present disclosure provides methods of treating facial pain or headache disorder by administering a therapeutically effective amount of a tryptamine to a patient in need thereof. In embodiments, the tryptamine is 5-methoxy-N,N-dimethyltryptamine (5-MeO-DMT) or a prodrug or a pharmaceutically acceptable salt thereof, or 5-hydroxy-N,N-dimethyltryptamine (5-OH-DMT) or a prodrug or a pharmaceutically acceptable salt thereof.

In embodiments, the disclosure provides methods of treating an acute headache in a patient in need thereof, the method comprising: administering a therapeutically effective amount of a tryptamine to the patient, wherein the tryptamine is 5-methoxy-N,N-dimethyltryptamine (5-MeO-DMT) or a prodrug or a pharmaceutically acceptable salt thereof, or 5-hydroxy-N,N-dimethyltryptamine (5-OH-DMT) or a prodrug or a pharmaceutically acceptable salt thereof.

In embodiments, the present disclosure provides methods of treating facial pain. In embodiments, the facial pain is orofacial pain or craniofacial pain. In embodiments, the facial pain is selected from the group consisting of temporomandibular disorder (TMD) associated pain, temporomandibular disorder (TMD) associated pain, neuralgias, trigeminal neuraligia, glossopharyngeal neuralgia, sphenopalatine ganglion neuralgia, postherpetic neuralgia, Sluder's neuralgia, mental nerve neuralgia, post-injury associated pain, burning mouth syndrome associated pain, persistent idiopathic associated pain, atypical odontalgia associated pain, mucosal ulceration associated pain, ulceration associated pain, traumatic ulceration associated pain, immunologic ulceration associated pain, infective ulceration associated pain, erosive ulceration associated pain, vesiculobullous associated pain, psychosomatic related pain, sinonasal related pain, rhinosinusitis associated pain, salivary gland disease associated pain, cardiac toothache associated pain, eagle syndrome associated pain, myofascial related pain, muscle spasm associated pain, dental related pain, craniofacial pain syndrome, temporal tendonitis associated pain, ernest syndrome associated pain, hamular bursitis associated pain, occipital neuritis associated pain, cervicalgia associated pain, fibromyalgia associated pain, trigeminal autonomic cephalalgias associated pain, cranial neuropathies associated pain, and pain associated with headache (e.g., cluster headache, tension type headache, neoplastic headache, aneurysm headache, and migraine).

In embodiments, the facial pain is selected from the group consisting of neuralgias, trigeminal neuraligia, glossopharyngeal neuralgia, sphenopalatine ganglion neuralgia, postherpetic neuralgia, Sluder's neuralgia, and mental nerve neuralgia.

In embodiments, the facial pain is a trigeminal neuraligia.

In embodiments, the headache is a migraine, classic migraine, migraine disorder, migraine without aura, migraine with aura, chronic migraine, acute migraine, episodic migraine, vestibular migraine, menstrual migraine, muscular character of migraine, headache, acute headache, chronic headache, tension type headache, medication overuse headache, cluster headache, post traumatic headache, postdural puncture headache, chronic cluster headache, episodic cluster headache, infrequent episodic tension-type headache, frequent episodic tension-type headache, or chronic tension-type headache. In embodiments the facial pain is a chronic pain. In embodiments the headache is an acute headache.

In embodiments, the headache is an acute headache or an acute migraine.

In embodiments, the headache is a chronic headache or a chronic migraine.

In embodiments, the headache is a cluster headache episode.

In embodiments, the tryptamine is 5-MeO-DMT or a prodrug or a pharmaceutically acceptable salt thereof. In embodiments, the method comprises administering about 0.25 mg to about 50 mg of 5-MeO-DMT to a patient in need thereof. In embodiments, about 0.25 mg to about 36 mg of 5-MeO-DMT is administered to a patient in need thereof.

In embodiments, the tryptamine is 5-OH-DMT or a prodrug or a pharmaceutically acceptable salt thereof. In embodiments, about 0.25 mg to about 50 mg of 5-OH-DMT is administered to a patient in need thereof.

In embodiments, the tryptamine is administered by insufflation, intranasally, orally, subcutaneously, sublingually, buccally, or by inhalation.

In embodiments, the method comprises administering a single dose of the tryptamine to the patient. In embodiments, the method further comprises administering a second dose tryptamine to the patient about 30 minutes after the first administration, wherein the second dose is administered if the first dose is not therapeutically effective. In embodiments, the tryptamine is administered at the onset of the acute headache. In embodiments, the administration reduces the patient's pain associated with the acute headache.

### BRIEF DESCRIPTION OF THE DRAWINGS

FIG. 1 shows an overview of the study design as described in Example 2.

FIG. 2 shows a bar graph of facial withdrawal scores on Day 1, measured 24 h after the final exposure to repeated stress. Values are mean±sem of the summed facial withdrawal scores for each treatment group. ***p<0.001 versus G2. G1: Naïve 0.9% sterile saline; G2: Model 0.9% sterile saline; G3: 0.9% sterile saline/sumatriptan 1 mg/kg; G4: 0.9% sterile saline/5-OH-DMT 3 mg/kg; G5: 0.9% sterile saline/5-OH-DMT 10 mg/kg; G6: 0.9% sterile saline/5-OH-DMT 30 mg/kg; G7: 5-OH-DMT 3 mg/kg/0.9% sterile saline; G8: 5-OH-DMT 10 mg/kg/0.9% sterile saline; G9:5-OH-DMT 30 mg/kg/0.9% sterile saline.

FIG. 3 shows a bar graph of facial withdrawal scores on Day 10 after exposure to repeated stress, measured prior to the 1^{st} test article injection. Values are mean±sem of the summed facial withdrawal scores for each treatment group. G1: Naïve 0.9% sterile saline; G2: Model 0.9% sterile saline; G3: 0.9% sterile saline/sumatriptan 1 mg/kg; G4: 0.9% sterile saline/5-OH-DMT 3 mg/kg; G5: 0.9% sterile saline/5-OH-DMT 10 mg/kg; G6: 0.9% sterile saline/5-OH-DMT 30 mg/kg; G7: 5-OH-DMT 3 mg/kg/0.9% sterile saline; G8: 5-OH-DMT 10 mg/kg/0.9% sterile saline; G9:5-OH-DMT 30 mg/kg/0.9% sterile saline.

FIG. 4 shows a bar graph of facial withdrawal scores on Day 11 after exposure to repeated stress, measured 3 h following SNP administration. Values are mean±sem of the summed facial withdrawal scores for each treatment group. ***p<0.001 versus G2 Model 0.9% sterile saline group. G1: Naïve 0.9% sterile saline; G2: Model 0.9% sterile saline; G3: 0.9% sterile saline/sumatriptan 1 mg/kg; G4: 0.9% sterile saline/5-OH-DMT 3 mg/kg; G5: 0.9% sterile saline/5-OH-DMT 10 mg/kg; G6: 0.9% sterile saline/5-OH-DMT 30 mg/kg; G7: 5-OH-DMT 3 mg/kg/0.9% sterile saline; G8: 5-OH-DMT 10 mg/kg/0.9% sterile saline; G9:5-OH-DMT 30 mg/kg/0.9% sterile saline.

FIG. 5 shows a bar graph of facial withdrawal scores on Day 12 after exposure to repeated stress, measured 1 h following test article injection #2 administration. Values are mean±sem of the summed facial withdrawal scores for each treatment group. ***p<0.001 versus G2 Model 0.9% sterile saline group. G1: Naïve 0.9% sterile saline; G2: Model 0.9% sterile saline; G3: 0.9% sterile saline/sumatriptan 1 mg/kg; G4: 0.9% sterile saline/5-OH-DMT 3 mg/kg; G5: 0.9% sterile saline/5-OH-DMT 10 mg/kg; G6: 0.9% sterile saline/5-OH-DMT 30 mg/kg; G7: 5-OH-DMT 3 mg/kg/0.9% sterile saline; G8: 5-OH-DMT 10 mg/kg/0.9% sterile saline; G9:5-OH-DMT 30 mg/kg/0.9% sterile saline.

### DETAILED DESCRIPTION

### Definitions

For convenience, certain terms employed in the specification, examples and claims are collected here. Unless defined otherwise, all technical and scientific terms used in this disclosure have the same meanings as commonly understood by one of ordinary skill in the art to which this disclosure belongs.

The term “carrier” as used herein encompasses carriers, excipients, and diluents, meaning a material, composition or vehicle, such as a liquid or solid filler, diluent, excipient, solvent or encapsulating material involved in carrying or transporting a pharmaceutical agent from one organ, or portion of the body, to another organ or portion of the body.

The term “disorder” is used in this disclosure to mean, and is used interchangeably with, the terms disease, condition, or illness, unless otherwise indicated.

The terms “effective amount” and “therapeutically effective amount” are used interchangeably in this disclosure and refer to an amount of a compound, or prodrug thereof, or a salt thereof, that, when administered to a patient, is capable of achieving the intended result. For example, an effective amount of a salt of 5-OH-DMT is that amount that is required to reduce at least one symptom of headache or facial pain in a patient. The actual amount that comprises the “effective amount” or “therapeutically effective amount” will vary depending on a number of conditions including, but not limited to, the severity of the disorder, the size and health of the patient, and the route of administration.

The phrase “pharmaceutically acceptable” as used herein refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The term “prodrug” as used herein refers to a derivative of the compound (e.g., 5-MeO-DMT or 5-OH-DMT) containing a functional group (such as an ester or amide) that is capable of releasing the compound when the prodrug form is administered to a patient. In embodiments, the functional group that releases 5-MeO-DMT from the 5-MeO-DMT prodrug is attached to the indole nitrogen atom of 5-MeO-DMT. In embodiments, the functional group that releases 5-OH-DMT from the 5-OH-DMT prodrug is attached to the indole nitrogen atom of 5-OH-DMT. In embodiments, the functional group that releases 5-OH-DMT from the 5-OH-DMT prodrug is attached to the phenolic oxygen atom of 5-OH-DMT. In embodiments, the 5-MeO-DMT prodrug or 5-OH-DMT prodrug is a compound as described in U.S. Patent Publication No. 20230041584, which is hereby incorporated by reference in its entirety.

The term “salts” as used herein embraces pharmaceutically acceptable salts commonly used to form addition salts of free bases. The nature of the salt is not critical, provided that it is pharmaceutically acceptable. The term “salts” also includes solvates of addition salts, such as hydrates, as well as polymorphs of addition salts. Suitable pharmaceutically acceptable acid addition salts can be prepared from an inorganic acid or from an organic acid.

The term “treating” as used herein with regard to a patient, refers to improving at least one symptom of the patient's headache (for example, headache or facial pain). Treating can be improving, or at least partially ameliorating a disorder.

As used herein, “therapeutically-effective dose” means a dose sufficient to achieve the intended therapeutic purpose, such as, to alleviate a sign or symptom of a disease or disorder in a patient.

The term “therapeutic effect” as used herein refers to a desired or beneficial effect provided by the method and/or the composition. For example, the method for treating headache provides a therapeutic effect when the method reduces at least one symptom of headache in a patient.

The term “prophylactically preventing” as used herein with regard to a patient, refers to preventing the occurrence or reoccurrence of a patient's symptoms (such as headache).

### 5-MeO-DMT and 5-OH-DMT

The present disclosure provides method of treating facial pain or headache by administering 5-methoxy-dimethyltryptamine (5-MeO-DMT) or a prodrug or a pharmaceutically acceptable salt thereof or 5-hydroxy-dimethyltryptamine (5-OH-DMT) or a prodrug or a pharmaceutically acceptable salt thereof.

5-Methoxy-dimethyltryptamine (5-MeO-DMT) is a natural psychoactive indolealkylamine found in toad venom of Bufo alvarius and a number of plants. The compound has been used historically by indigenous cultures and produces a range of rapid and short-lasting subjective effects, including altered visual, auditory and time perceptions, euphoria and interconnectedness, as well as physical effects when consumed by smoking or the intravenous route (Shulgin and Shulgin, 1997; Ott, 2001b; Barsuglia et al., 2018; Uthaug et al., 2020). Inhaled 5-MeO-DMT administered in a naturalistic group setting to individuals using the compound for spiritual purposes was associated with improvements in self-reported symptoms of depression and anxiety (Davis et al., 2019). Interestingly, a recent human field study reported that single inhalation of vapor from dried toad secretion containing 5-MeO-DMT (and very low amounts of its active metabolite, 5-OH-DMT) produced improvements in affect and cognition at 24 hours, which was maintained at 4 weeks following administration (Uthaug et al., 2019). In its Form F-1 filing, GH Research PLC reported completion of a single-dose Phase 1 clinical trial of their proprietary inhaled 5-MeO-DMT preparation (GH001) at 12 and 18 mg in 16 patients with treatment-resistant depression, which found clinical remission in 2 patients on day 7 after receiving 12 mg and 1 patient on day 7 after receiving 18 mg and a positive clinical response in 1 patient on day 7 after receiving 18 mg. Apparent rapid onset and maintained therapeutic-like effects following single administration also have been reported for other psychedelic drugs, such as psilocybin to produce clinical antimigraine and antidepressant effects and MDMA to produce clinical anxiolytic efficacy (Schindler et al., 2020; Carhart-Harris et al., 2017; Mithoefer et al., 2010).

5-MeO-DMT is inactivated mainly by monoamine oxidase A and, to a more limited extent, O-demethylated by CYP2D6 to an active metabolite, 5-OH-DMT or bufotenine (Shen et al., 2010). 5-OH-DMT also is found naturally in toads and plants. Reports of its rapid and short-lived psychoactivity include altered visual perceptions and physical effects, such as body area tightness, facial flushing and nausea, when consumed intravenously, intranasally or sublingually (Shulgin and Shulgin, 1997; McBride, 2000; Ott, 2001a).

5-MeO-DMT is a potent agonist across many serotonin (5-HT) receptor subtypes (e.g., 5-HT1A, 1B, 2A, 2B, 6) and also binds to multiple dopaminergic and noradrenergic alpha2 receptor subtypes, serotonin and norepinephrine transporters, and imidazolinel receptors (Ray, 2010; Cameron et al., 2021). 5-MeO-DMT induces the head twitch response in mice, indicative of in vivo 5-HT2A receptor activation, while agonism at 5-HT1A receptors also is involved in its behavioral effects in animals (Winter et al., 1999; Halberstadt et al., 2011; Cameron et al., 2021; Dunlap et al., 2020). 5-MeO-DMT stimulates neuroplastic changes, as evidenced by increased neurite outgrowth in cortical cultures, altered protein expression in human cerebral organoids, and enhanced proliferation and maturation of hippocampal dentate gyrus granule cells in adult mice (Dunlap et al., 2020; Dakic et al., 2017; Lima da Cruz et al., 2018). Of potential relevance to the described method of treatment of a painful condition, such as headache, 5-MeO-DMT exhibited acute analgesic effects in rats (Archer et al., 1986). The active metabolite 5-OH-DMT also binds to multiple subtypes of 5-HT receptors, including binding and stimulation of 5-HT2A receptor signaling with increased potency relative to 5-MeO-DMT (Lyon et al., 1988; Roth et al., 1997; McBride, 2000). 5-OH-DMT induced the head twitch response in mice (ED50=15 mg/kg, subcutaneous), indicative of in vivo 5-HT2A receptor activation (Come and Pickering, 1967). In addition, 5-OH-DMT exhibited acute anti-inflammatory and analgesic effects in mice (Wang et al., 2021).

5-MeO-DMT has reported psychoactive effects in humans in the range of 6-30 mg when smoked, 0.25-3.1 mg when administered intravenously, or ˜10 mg sublingually (TiHKAL, Shulgin & Shulgin, 1997; Shen et al., 2010). Upon insufflation of 10 mg 5-MeO-DMT, initial effects were reported at 3-4 minutes, peaking at 35-40 minutes and terminating by 60-70 minutes (Ott, 2001b). There are reports of psychoactive effects following oral administration (activity at 10-25 mg following pretreatment with a monoamine oxidase inhibitor (e.g., harmaline) or 30 mg when encapsulated; TiHKAL, Shulgin & Shulgin, 1997; Ott, 2001b), but the reduced potency and anticipated slower pharmacokinetic profile of the oral route would not be preferred for the treatment of acute headache. Similarly, 5-OH-DMT induces psychoactive effects in humans following administration via inhalation of vapor, intranasally and sublingually, with lower potency following the oral (encapsulated) route (Ott, 2001a). Upon insufflation of 40 mg 5-OH-DMT, initial effects were reported at 5 minutes, peaking at 35-40 minutes with short-lived aftereffects for up to 90 minutes (Ott, 2001a).

### Formulations

The methods of the present invention can employ various formulations for administration to patients, e.g., humans in unit dosage forms, such as tablets, capsules, pills, powders, granules, sterile parenteral solutions or suspensions (e.g., intramuscular (IM), subcutaneous (SC) and intravenous (IV)), transdermal patches, and oral solutions or suspensions, and oil-water emulsions containing suitable quantities of a tryptamine as described herein.

Oral pharmaceutical dosage forms can be either solid or liquid. The solid dosage forms can be tablets, capsules, granules, films (e.g., buccal films) and bulk powders. Types of oral tablets include compressed, chewable lozenges and tablets, which can be enteric-coated, sugar-coated or film-coated. Capsules can be hard or soft gelatin capsules, while granules and powders can be provided in non-effervescent or effervescent form with the combination of other ingredients known to those skilled in the art. In some embodiments, the present oral dosage forms may include orally disintegrating tablets.

In embodiments, the disclosure provides a pharmaceutical composition comprising a tryptamine and one or more pharmaceutically acceptable carriers or excipients.

In embodiments, the pharmaceutical composition comprises from about 0.1 mg to about 100 mg of tryptamine. In embodiments, the pharmaceutical composition comprises from about 2 to about 40 mg, from about 2 to about 10 mg, from about 5 to about 30 mg, from about 5 to about 15 mg, or from about 20 to about 30 mg of a tryptamine. In embodiments, the pharmaceutical composition comprises about 1 mg, about 5 mg, about 10 mg, or about 25 mg of the tryptamine.

In embodiments, the pharmaceutical composition comprises 5-MeO-DMT. In embodiments, the pharmaceutical composition comprises 5-OH-DMT.

### Methods of the Disclosure

In embodiments, the present disclosure provides methods of treating facial pain by administering a therapeutically effective amount of a tryptamine to a patient in need thereof. In embodiments, the present disclosure provides methods of treating headache by administering a therapeutically effective amount of a tryptamine to a patient in need thereof. In embodiments, the tryptamine is 5-methoxy-N,N-dimethyltryptamine (5-MeO-DMT) or a prodrug or a pharmaceutically acceptable salt thereof, or 5-hydroxy-N,N-dimethyltryptamine (5-OH-DMT) or a prodrug or a pharmaceutically acceptable salt thereof. In embodiments, the tryptamine is administered in a pharmaceutical composition as described herein.

In embodiments, the facial pain is orofacial pain and/or craniofacial pain. In embodiments, the orofacial pain and craniofacial pain. In embodiments, the facial pain includes but is not limited to, temporomandibular disorder (TMD) associated pain, temporomandibular joint disorder (TMJ) associated pain, neuralgias (e.g., trigeminal neuraligia, glossopharyngeal neuralgia, sphenopalatine ganglion neuralgia, postherpetic neuralgia, Sluder's neuralgia, mental nerve neuralgia), post-injury associated pain, burning mouth syndrome associated pain, persistent idiopathic associated pain (i.e., atypical facial pain), atypical odontalgia associated pain, mucosal ulceration associated pain, ulceration associated pain, traumatic ulceration associated pain, immunologic ulceration facial pain, infective ulceration associated pain, erosive ulceration associated pain, vesiculobullous (e.g., oral ulceration, aphthous stomatitis, erosive oral lichen planus) associated pain, psychosomatic related pain, sinonasal related pain, rhinosinusitis associated pain, salivary gland disease (e.g., sialadenitis and sialoithiasis) associated pain, cardiac toothache associated pain, eagle syndrome associated pain, myofascial related pain, muscle spasm associated pain, dental related pain, craniofacial pain syndrome, temporal tendonitis associated pain, ernest syndrome associated pain, hamular bursitis associated pain, occipital neuritis associated pain, cervicalgia associated pain, fibromyalgia associated pain, trigeminal autonomic cephalalgias associated pain, cranial neuropathies associated pain, and pain associated with headache (e.g., cluster headache, tension type headaches, neoplastic headache, aneurysm headache, migraine).

In embodiments, the facial pain is caused by post-injury, burning mouth syndrome, persistent idiopathic, atypical odontalgia, mucosal ulceration, ulceration, traumatic ulceration, immunologic ulceration, infective ulceration, erosive ulceration, vesiculobullous, psychosomatic related pain, sinonasal related pain, rhinosinusitis associated pain, salivary gland disease, cardiac toothache, eagle syndrome, myofascial related pain, muscle spasm, dental related pain, craniofacial pain syndrome, temporal tendonitis, ernest syndrome, hamular bursitis, occipital neuritis, cervicalgia, fibromyalgia, trigeminal autonomic cephalalgias, cranial neuropathies, or headache (e.g., cluster headache, tension type headaches, neoplastic headache, aneurysm headache, and migraine).

In embodiments, the facial pain arises from cancer, infection, a burn, a laceration, a broken bone, a toothache, temporomandibular joint disorder (TMJ), a dental procedure, a medical surgical procedure, or cosmetic procedure.

In embodiments, the facial pain is temporomandibular disorder (TMD) associated pain, temporomandibular joint disorder (TMJ) associated pain, or neuralgias (e.g., trigeminal neuraligia, glossopharyngeal neuralgia, sphenopalatine ganglion neuralgia, postherpetic neuralgia, Sluder's neuralgia, mental nerve neuralgia).

In embodiments, the facial pain is trigeminal neuralgia (TN). In embodiments, the facial pain is associated with migraine or headache. In embodiments, the facial pain is associated with atypical facial pain or mononeuropathies. In some embodiments, the facial pain is double- or one-sided. In embodiments, the facial pain is constant, periodic or both. In embodiments, the duration of periodic facial pain varies from a few seconds to several minutes or hours. In embodiments, common everyday activities such as eating, talking, shaving and brushing teeth triggers or worsens the facial pain.

In embodiments, the methods of the present disclosure are used to treat, prevent, and/or alleviate facial pain, such as facial pain in a patient suffering from a headache and/or migraine. In embodiments, the headache and migraine related conditions is defined by ICD-11 MMS, for example, migraine, classic migraine, migraine disorder, migraine without aura, migraine with aura, chronic migraine, acute migraine, episodic migraine, vestibular migraine, menstrual migraine, muscular character of migraine, headache, acute headache, chronic headache, tension type headache, medication overuse headache, cluster headache, post traumatic headache, postdural puncture headache, chronic cluster headache, episodic cluster headache, infrequent episodic tension-type headache, frequent episodic tension-type headache, and chronic tension-type headache. In embodiments the facial pain is a chronic pain. In embodiments the facial pain is an acute pain.

In embodiments, the methods of the present disclosure are used to treat headache disorders including migraines, classic migraines, migraine disorders, headaches, chronic migraines, migraines with aura, episodic migraines, acute migraines, vestibular migraines, tension type headaches, medication overuse headaches, menstrual migraines, cluster headaches, chronic headaches, post traumatic headaches, postdural puncture headaches, chronic cluster headaches, and episodic cluster headaches. In embodiments, the headache and migraine related conditions are defined by ICD-11 MMS, for example, migraine, classic migraine, migraine disorder, migraine without aura, migraine with aura, chronic migraine, acute migraine, episodic migraine, vestibular migraine, menstrual migraine, muscular character of migraine, headache, acute headache, chronic headache, tension type headache, medication overuse headache, cluster headache, post traumatic headache, postdural puncture headache, chronic cluster headache, episodic cluster headache, infrequent episodic tension-type headache, frequent episodic tension-type headache, and chronic tension-type headache. In embodiments the facial pain is a chronic pain. In embodiments the facial pain is an acute pain.

In embodiments, the present disclosure provides a method of treating an acute headache in a patient in need thereof, the method comprising: administering a therapeutically effective amount of a tryptamine to a patient in need thereof, wherein the tryptamine is 5-methoxy-N,N-dimethyltryptamine (5-MeO-DMT) or a prodrug or a pharmaceutically acceptable salt thereof, or 5-hydroxy-N,N-dimethyltryptamine (5-OH-DMT) or a prodrug or a pharmaceutically acceptable salt thereof.

In embodiments, the acute headache is an acute migraine. In embodiments, the acute headache is a cluster headache episode.

In embodiments, the present disclosure provides methods of treatment of an acute single migraine and/or cluster headache episode by administering a single dose of 5-MeO-DMT and/or 5-OH-DMT to a patient in need thereof.

According to the methods of the present disclosure, the tryptamine (e.g., 5-MeO-DMT) may be administered to the patient in need thereof using any suitable route of administration known to those skilled in the art, including oral, parenteral (e.g., intravenous, subcutaneous, intradermal, intramuscular, intradermal, intrapleural, intracerebral, and intra-articular), topical (e.g., to both skin and mucosal surfaces, including airway surfaces, and transdermal administration), inhalation (e.g., via an aerosol), rectal, transmucosal, intranasal, buccal, sublingual, vaginal, intrathecal, intraocular, and transdermal. In embodiments, the tryptamine is administered by insufflation. In embodiments, the tryptamine is administered by inhalation. In embodiments, the tryptamine is intranasally administered. In embodiments, the tryptamine is orally administered. In embodiments, the tryptamine is subcutaneously administered. In embodiments, the tryptamine is sublingually administered. In embodiments, the tryptamine is buccally administered.

In embodiments, the present disclosure provides methods of treating and prophylactically preventing reoccurrence of acute headaches by administering a therapeutically effective amount of a tryptamine to a patient in need thereof. In embodiments, the tryptamine is 5-methoxy-N,N-dimethyltryptamine (5-MeO-DMT) or a prodrug or a pharmaceutically acceptable salt thereof, or 5-hydroxy-N,N-dimethyltryptamine (5-OH-DMT) or a prodrug or a pharmaceutically acceptable salt thereof. In embodiments, the tryptamine is administered in a pharmaceutical composition as described herein.

In embodiments, the present disclosure provides methods of treating an acute headache and prophylactically preventing reoccurrence of acute headaches in a patient in need thereof, the methods comprising administering a therapeutically effective amount of a tryptamine to the patient, wherein the patient experiences at least 2 migraine headaches a month during the 3 months prior to administration.

In embodiments, the present disclosure provides methods of treating an acute headache and prophylactically preventing reoccurrence of acute headaches in a patient in need thereof, the methods comprising administering a first dose of a tryptamine to the patient; determining whether the first dose of the tryptamine alleviated the patient's headache; and if the first dose did not alleviate the patient's headache, administering a second dose of the tryptamine after administering the first dose, wherein the patient experiences at least 2 migraine headaches a month during the 3 months prior to administration.

In embodiments, the methods comprise administering at least 2 doses of a tryptamine to the patient. In embodiments, the methods comprising administering at least 2, at least 3, at least 4, at least 5, at least 6, at least 7, at least 8, at least 9, or at least 10 doses of a tryptamine to the patient.

In embodiments, the methods comprise determining whether the first dose of the tryptamine alleviated the patient's headache; and if the first dose did not alleviate the patient's headache, administering a second dose of the tryptamine after administering the first dose. In embodiments, determining whether the first dose of the tryptamine alleviated the patient's headache comprises assessing if the patient has experienced a reduction in headache symptoms. In embodiments, headache symptoms include nausea, vomiting, sensitivity to light, dizziness, loss of balance, aura, or sensitivity to sound.

In embodiments, a subsequent dose (e.g., second, third, fourth, fifth, sixth, seventh, eighth, ninth, or tenth) of the tryptamine is administered to the patient on an “as-needed basis,” for example, when the patient experiences a migraine, the onset of a migraine, or migraine symptoms, the patient is administered a subsequent dose of the tryptamine. In embodiments, the patient is administered a subsequent dose of the tryptamine about 10 min, about 15 min, about 20 min, about 25 min, about 30 min, about 35 min, about 40 min, about 45 min, about 50 min, about 55 min, about 1 hour, about 2 hours, about 4 hours, about 6 hours, about 8 hours, about 10 hours, about 12 hours, about 14 hours, about 16 hours, about 18 hours, about 20 hours, about 22 hours, or about 1 day after administration of a previous dose of the tryptamine. In embodiments, a subsequent dose of the tryptamine is administered about 1 day, about 2 days, about 3 days, about 4 days, about 5 days, about 6 days, or about 1 week after administration of a previous dose of a tryptamine.

In embodiments, the patient experiences at least 3 migraine headaches a month during the 3 months prior to administration. In embodiments, the patient experiences at least 4 migraine headaches a month during the 3 months prior to administration. In embodiments, the patient experiences at least 5 migraine headaches a month during the 3 months prior to administration. In embodiments, the patient experiences at least 6 migraine headaches a month during the 3 months prior to administration. In embodiments, the patient experiences at least 7 migraine headaches a month during the 3 months prior to administration. In embodiments, the patient experiences at least 8 migraine headaches a month during the 3 months prior to administration. In embodiments, the patient experiences at least 9 migraine headaches a month during the 3 months prior to administration. In embodiments, the patient experiences at least 10 migraine headaches a month during the 3 months prior to administration.

In embodiments, the patient exhibits at least about a 30% reduction in a mean monthly number of migraine headaches after administration of the tryptamine. In embodiments, the patient exhibits at least about a 30%, at least about a 40%, at least about a 50%, at least about a 60%, at least about a 70%, at least about an 80%, at least about a 90%, or at least about a 100% reduction in the mean monthly number of migraine headaches after administration of the tryptamine.

In embodiments, the patient exhibits at least about a 30% reduction in a mean monthly number of migraine headaches after administration of a first, second, third, fourth, fifth, sixth, seventh, eighth, ninth, or tenth dose of the tryptamine. In embodiments, the patient exhibits at least about a 30%, at least about a 40%, at least about a 50%, at least about a 60%, at least about a 70%, at least about an 80%, at least about a 90%, or at least about a 100% reduction in the mean monthly number of migraine headaches after administration of a first, second, third, fourth, fifth, sixth, seventh, eighth, ninth, or tenth dose of the tryptamine.

In embodiments, the patient exhibits at least about a 30% reduction in a mean monthly number of migraine headaches after administration of a first dose of the tryptamine. In embodiments, the patient exhibits at least about a 30%, at least about a 40%, at least about a 50%, at least about a 60%, at least about a 70%, at least about an 80%, at least about a 90%, or at least about a 100% reduction in the mean monthly number of migraine headaches after administration of a first dose of the tryptamine.

In embodiments, the patient exhibits at least about a 30% reduction in a mean monthly number of migraine headaches after administration of a second dose of the tryptamine. In embodiments, the patient exhibits at least about a 30%, at least about a 40%, at least about a 50%, at least about a 60%, at least about a 70%, at least about an 80%, at least about a 90%, or at least about a 100% reduction in the mean monthly number of migraine headaches after administration of a second dose of the tryptamine.

In embodiments, the patient exhibits at least about a 30% reduction in a mean monthly number of migraine headaches about 10 min, about 15 min, about 20 min, about 25 min, about 30 min, about 35 min, about 40 min, about 45 min, about 50 min, about 55 min, about 1 hour, about 2 hours, about 4 hours, about 6 hours, about 8 hours, about 10 hours, about 12 hours, about 14 hours, about 16 hours, about 18 hours, about 20 hours, about 22 hours, about 1 day, about 2 days, about 3 days, about 4 days, about 5 days, about 6 days, or about 1 week after administration of the tryptamine.

In embodiments, the patient exhibits at least about a 30%, at least about a 40%, at least about a 50%, at least about a 60%, at least about a 70%, at least about an 80%, at least about a 90%, or at least about a 100% reduction in the mean monthly number of migraine headaches about 10 min, about 15 min, about 20 min, about 25 min, about 30 min, about 35 min, about 40 min, about 45 min, about 50 min, about 55 min, about 1 hour, about 2 hours, about 4 hours, about 6 hours, about 8 hours, about 10 hours, about 12 hours, about 14 hours, about 16 hours, about 18 hours, about 20 hours, about 22 hours, about 1 day, about 2 days, about 3 days, about 4 days, about 5 days, about 6 days, or about 1 week after administration of the tryptamine.

In embodiments, the patient experiences less than 3 migraine headaches a month after administration of the tryptamine. In embodiments, the patient experiences less than 2 migraine headaches a month after administration of the tryptamine. In embodiments, the patient experiences less than 1 migraine headache a month after administration of the tryptamine. In embodiments, the patient experiences no migraine headaches after administration of the tryptamine.

In embodiments, the patient experiences less than 3 migraine headaches a month after administration of one, two, three, four, five, six, seven, eight, nine, or ten doses of the tryptamine. In embodiments, the patient experiences less than 2 migraine headaches a month after administration of one, two, three, four, five, six, seven, eight, nine, or ten doses of the tryptamine. In embodiments, the patient experiences less than 1 migraine headache a month after administration of one, two, three, four, five, six, seven, eight, nine, or ten doses the tryptamine. In embodiments, the patient experiences no migraine headaches after administration of one, two, three, four, five, six, seven, eight, nine, or ten doses the tryptamine.

In embodiments, the patient experiences less than 3 migraine headaches a month after administration of a first dose of the tryptamine. In embodiments, the patient experiences less than 2 migraine headaches a month after administration of a first dose of the tryptamine. In embodiments, the patient experiences less than 1 migraine headache a month after administration of a first dose of the tryptamine. In embodiments, the patient experiences no migraine headaches after administration of a first dose of the tryptamine.

In embodiments, the patient experiences less than 3 migraine headaches a month after administration of a second dose of the tryptamine. In embodiments, the patient experiences less than 2 migraine headaches a month after administration of a second dose of the tryptamine. In embodiments, the patient experiences less than 1 migraine headache a month after administration of a second dose of the tryptamine. In embodiments, the patient experiences no migraine headaches after administration of a second dose of the tryptamine.

### Dosing

In embodiments, the methods of the present disclosure comprise administering about 0.1 mg to about 100 mg, about 1 mg to about 50 mg, or about 5 mg to about 30 mg of a tryptamine (e.g., 5-MeO-DMT or 5-OH-DMT) to a patient in need thereof. In embodiments, the methods of the present disclosure comprise administering about 0.10 mg, about 0.15 mg, about 0.20 mg, about 0.25 mg, about 0.30 mg, about 0.35 mg, about 0.40 mg, about 0.45 mg, about 0.50 mg, about 0.55 mg, about 0.60 mg, about 0.65 mg, about 0.70 mg, about 0.75 mg, about 0.80 mg, about 0.85 mg, about 0.90 mg, about 0.95 mg, about 1 mg, about 2 mg, about 3 mg, about 4 mg, about 5 mg, about 6 mg, about 7 mg, about 8 mg, about 9 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg or about 100 mg of a tryptamine (e.g. 5-MeO-DMT) to a patient in need thereof.

In embodiments, the method of the present disclosure comprises administering a therapeutically effective amount of 5-MeO-DMT or a prodrug or a pharmaceutically acceptable salt thereof to a patient in need thereof.

In embodiments, about 0.25 mg to about 50 mg of 5-MeO-DMT is administered to a patient in need thereof. In embodiments, about 6 mg to about 30 mg of 5-MeO-DMT is administered to a patient in need thereof. In embodiments, about 10 mg of 5-MeO-DMT is administered to a patient in need thereof. In embodiments, about 0.25 mg to about 3 mg of 5-MeO-DMT is administered to a patient in need thereof. In embodiments, about 0.25 mg, about 0.30 mg, about 0.35 mg, about 0.40 mg, about 0.45 mg, about 0.50 mg, about 0.55 mg, about 0.60 mg, about 0.65 mg, about 0.70 mg, about 0.75 mg, about 0.80 mg, about 0.85 mg, about 0.90 mg, about 0.95 mg, about 1 mg, about 2 mg, about 3 mg, about 4 mg, about 5 mg, about 6 mg, about 7 mg, about 8 mg, about 9 mg, about 10 mg, about 15 mg, about 20 mg, about 25 mg, about 30 mg, about 35 mg, about 40 mg, about 45 mg or about 50 mg of 5-MeO-DMT is administered to a patient in need thereof.

In embodiments, the method of the present disclosure comprises administering a therapeutically effective amount of 5-OH-DMT or a prodrug or a pharmaceutically acceptable salt thereof to a patient in need thereof.

In embodiments, about 0.25 mg to about 50 mg of 5-OH-DMT is administered to a patient in need thereof. In embodiments, about 6 mg to about 30 mg of 5-OH-DMT is administered to a patient in need thereof. In embodiments, about 10 mg of 5-OH-DMT is administered to a patient in need thereof. In embodiments, about 0.25 mg to about 3 mg of 5-OH-DMT is administered to a patient in need thereof. In embodiments, about 0.25 mg, about 0.30 mg, about 0.35 mg, about 0.40 mg, about 0.45 mg, about 0.50 mg, about 0.55 mg, about 0.60 mg, about 0.65 mg, about 0.70 mg, about 0.75 mg, about 0.80 mg, about 0.85 mg, about 0.90 mg, about 0.95 mg, about 1 mg, about 2 mg, about 3 mg, about 4 mg, about 5 mg, about 6 mg, about 7 mg, about 8 mg, about 9 mg, about 10 mg, about 15 mg, about 20 mg, about 25 mg, about 30 mg, about 35 mg, about 40 mg, about 45 mg or about 50 mg is administered to a patient in need thereof.

In embodiments, the dosing frequency and dose amount per administration of the tryptamine (e.g., 5-MeO-DMT or 5-OH-DMT) are selected to provide therapeutic effects for the treatment of facial pain.

In embodiments, the dosing frequency and dose amount per administration of the tryptamine (e.g., 5-MeO-DMT or 5-OH-DMT) are selected to provide therapeutic effects for the treatment of an acute headache.

In embodiments, the dosing frequency and dose amount per administration of the tryptamine (e.g., 5-MeO-DMT or 5-OH-DMT) are selected to provide a reduction in incidence of headaches.

In embodiments, the dosing frequency and dose amount per administration of the tryptamine (e.g., 5-MeO-DMT or 5-OH-DMT) are selected to provide a reduction in the patient's pain associated with the acute headache.

In embodiments, the dosing frequency and dose amount per administration of the tryptamine (e.g., 5-MeO-DMT or 5-OH-DMT) are selected to provide one or more of the following: improved safety outcome, decreased adverse events, reduced headache frequency, reduced number of days with headache, reduced number of participants with adverse events, improved quality of life, improvement in headache impact test, reduction in headache intensity, improvement in migraine disability assessment, reduction in headache, reduction in migraine days, reduced use of rescue medication, reduced frequency of migraine attacks, reduction in headache severity, and improved patient satisfaction.

In embodiments, the tryptamine is administered at the onset of the acute headache.

In embodiments, the tryptamine is administered at the onset of the facial pain.

In embodiments, the tryptamine is administered in a single dose to a patient in need thereof.

In embodiments, the tryptamine is administered in multiple doses to a patient in need thereof. In some embodiments, the tryptamine is administered multiple doses at intervals that are selected to provide therapeutic effectiveness. In embodiments, a second dose of the tryptamine is administered to the patient about 30 minutes after the first administration, wherein the second dose is administered if the first dose is not therapeutically effective.

In embodiments, the same dose of a tryptamine is administered to a patient at each administration (e.g., 10 mg of 5-MeO-DMT is administered twice to a patient in need thereof). In embodiments, a different dose of a tryptamine is administered to a patient at each administration. In embodiments, the dose of a tryptamine administered to the patient is increased over time (i.e., dose escalation). In embodiments, the dose of a tryptamine administered to the patient is decreased over time (i.e., dose de-escalation).

### Clinical Outcomes

The methods of the present disclosure are used to treat facial pain in a patient. In embodiments, the efficacy of treatment is determined by performing pain assessment based on visual analog scale (VAS). It is a 10 cm horizontal or vertical line with word anchors at each end, such as “no pain” and “pain as bad as it could be”. A subject or patient is asked to make a mark on the line to represent pain intensity. This mark is converted to distance in either centimeters or millimeters from the “no pain” anchor to give a pain score that can range from 0-10 cm or 0-100 mm. The VAS may refer to an 11 point numerical pain rating scale wherein 0 equals “no pain” and 10 equals the “worst pain imaginable”. In embodiments, the efficacy of treatment is determined by performing pain assessment based on numerical rating scale (NRS) score.

In embodiments, the VAS score of the patient during the administration period is less than the VAS score of the patient prior to treatment. In embodiments, the VAS score of the patient during the administration period is at least about 10% less (for example, at least 15% less, at least 20% less, at least 25% less, at least 30% less, at least 35% less, at least 40% less, at least 45% less, at least 50% less, at least 55% less, at least 60% less, at least 65% less, at least 70% less, at least 75% less, at least 80% less, at least 85% less, at least 90% less, at least 95% less, or at least 100% less, including all values and subranges that lie therebetween) than the VAS score of the patient prior to treatment. In embodiments, the VAS score of the patient during the administration period is at least about 1 point less (e.g., 1, 1.5, 2, 2.5, 3, 3.5, 4, 4.5, 5, 5.5, 6, 6.5, 7, 7.5, 8, 8.5, 9, 9.5, or 10) than the VAS score of the patient prior to treatment.

### NUMBERED EMBODIMENTS OF THE DISCLOSURE

In addition to the disclosure above, the Examples below, and the appended claims, the disclosure sets forth the following numbered embodiments.

1. A method of treating facial pain or headache in a patient in need thereof comprising administering a therapeutically effective amount of a tryptamine comprising 5-methoxy-N,N-dimethyltryptamine (5-MeO-DMT), 5-hydroxy-N,N-dimethyltryptamine (5-OH-DMT) or prodrugs or pharmaceutically acceptable salts thereof.
2. The method of embodiment 1, wherein the facial pain is selected from the group consisting of temporomandibular disorder (TMD) associated pain, neuralgias, trigeminal neuraligia, glossopharyngeal neuralgia, sphenopalatine ganglion neuralgia, postherpetic neuralgia, Sluder's neuralgia, mental nerve neuralgia, post-injury associated pain, burning mouth syndrome associated pain, persistent idiopathic associated pain, atypical odontalgia associated pain, mucosal ulceration associated pain, ulceration associated pain, traumatic ulceration associated pain, immunologic ulceration associated pain, infective ulceration associated pain, erosive ulceration associated pain, vesiculobullous associated pain, psychosomatic related pain, sinonasal related pain, rhinosinusitis associated pain, salivary gland disease associated pain, cardiac toothache associated pain, eagle syndrome associated pain, myofascial related pain, muscle spasm associated pain, dental related pain, craniofacial pain syndrome, temporal tendonitis associated pain, ernest syndrome associated pain, hamular bursitis associated pain, occipital neuritis associated pain, cervicalgia associated pain, fibromyalgia associated pain, trigeminal autonomic cephalalgias associated pain, cranial neuropathies associated pain, and pain associated with headache.
3. The method of embodiment 2, wherein the facial pain is a trigeminal neuraligia.
4. The method of embodiment 1, wherein the headache is a migraine, classic migraine, migraine disorder, migraine without aura, migraine with aura, chronic migraine, acute migraine, episodic migraine, vestibular migraine, menstrual migraine, muscular character of migraine, headache, acute headache, chronic headache, tension type headache, medication overuse headache, cluster headache, post traumatic headache, postdural puncture headache, chronic cluster headache, episodic cluster headache, infrequent episodic tension-type headache, frequent episodic tension-type headache, or chronic tension-type headache.
5. The method of embodiment 4, wherein the headache is an acute headache.
6. The method of embodiment 4, wherein the headache is an acute migraine.
7. The method of embodiment 4, wherein the headache is a chronic headache or a chronic migraine.
8. The method of embodiment 4, wherein the headache is a cluster headache episode.
9. The method of embodiment 1, wherein the method comprises administering a therapeutically effective amount of 5-MeO-DMT or a prodrug or a pharmaceutically acceptable salt thereof to the patient.
10. The method of embodiment 9, wherein the method comprises administering between about 0.25 mg to about 50 mg of 5-MeO-DMT.
11. The method of embodiment 9, wherein the method comprises administering between about 0.25 mg to about 36 mg of 5-MeO-DMT.
12. The method of embodiment 9, wherein the method comprises administering between about 6 mg to about 30 mg of 5-MeO-DMT.
13. The method of embodiment 9, wherein the method comprises administering between about 0.25 mg to about 3 mg of 5-MeO-DMT.
14. The method of embodiment 9, wherein the method comprises administering about 10 mg of 5-MeO-DMT.
15. The method of embodiment 1, wherein the method comprises administering a therapeutically effective amount of 5-OH-DMT or a prodrug or a pharmaceutically acceptable salt thereof to the patient.
16. The method of embodiment 15, wherein the method comprises administering between about 0.25 mg to about 50 mg of 5-OH-DMT.
17. The method of embodiment 1, wherein the method comprises administering the tryptamine by insufflation.
18. The method of embodiment 1, wherein the method comprises administering the tryptamine intranasally.
19. The method of embodiment 1, wherein the method comprises administering the tryptamine orally.
20. The method of embodiment 1, wherein the method comprises administering the tryptamine subcutaneously.
21. The method of embodiment 1, wherein the method comprises administering the tryptamine sublingually.
22. The method of embodiment 1, wherein the method comprises administering the tryptamine buccally.
23. The method of embodiment 1, wherein the method comprises administering the tryptamine by inhalation.
24. The method of embodiment 1, wherein the method comprises administering a single dose of the tryptamine to the patient.
25. The method of embodiment 24, wherein the method further comprises administering a second dose of tryptamine to the patient about 30 minutes after a first administration if the first dose is not therapeutically effective.
26. The method of embodiment 1, wherein the method comprises administering the tryptamine at the onset of the facial pain or headache.
27. The method of embodiment 1, wherein administering the tryptamine reduces the visual analog scale (VAS) score by at least about 1 point compared to the VAS score of the patient prior to treatment.
28. A method of treating and prophylactically preventing reoccurrence of acute headaches in a patient in need thereof, the method comprising administering a therapeutically effective amount of a tryptamine comprising 5-methoxy-N,N-dimethyltryptamine (5-MeO-DMT), 5-hydroxy-N,N-dimethyltryptamine (5-OH-DMT) or prodrugs or pharmaceutically acceptable salts thereof to the patient, wherein the patient experiences at least 2 migraine headaches a month during the 3 months prior to administration.
29. The method of embodiment 28, comprising administering at least two doses of the tryptamine to the patient in need thereof.
30. The method of embodiment 28, comprising administering at least three doses of the tryptamine to the patient in need thereof.
31. The method of embodiment 29, wherein the at least two doses of the tryptamine are administered 30 minutes (min) apart.
32. The method of embodiment 30, wherein the at least three doses of the tryptamine are administered 30 min apart.
33. The method of embodiment 28, wherein the patient experiences at least 3 headaches a month during the 3 months prior to administration.
34. The method of embodiment 28, wherein the patient experiences at least 4 headaches a month during the 3 months prior to administration.
35. The method of embodiment 28, wherein the patient experiences at least 5 headaches a month during the 3 months prior to administration.
36. The method of embodiment 28, wherein the patient exhibits a 30% reduction in a mean monthly number of migraines after administration of the tryptamine.
37. The method of embodiment 28, wherein the migraine headache is an acute migraine.
38. The method of embodiment 28, wherein the migraine headache is a cluster headache episode.
39. The method of embodiment 28, comprising administering a therapeutically effective amount of 5-MeO-DMT or a prodrug or a pharmaceutically acceptable salt thereof to the patient.
40. The method of embodiment 39, comprising administering between about 0.25 mg to about 50 mg of 5-MeO-DMT.
41. The method of embodiment 39, comprising administering between about 0.25 mg to about 36 mg of 5-MeO-DMT.
42. The method of embodiment 39, comprising administering between about 6 mg to about 30 mg of 5-MeO-DMT.
43. The method of embodiment 39, comprising administering between about 0.25 mg to about 3 mg of 5-MeO-DMT.
44. The method of embodiment 39, comprising administering about 10 mg of 5-MeO-DMT.
45. The method of embodiment 28, comprising administering a therapeutically effective amount of 5-OH-DMT or a prodrug or a pharmaceutically acceptable salt thereof to the patient.
46. The method of embodiment 45, comprising administering between about 0.25 mg to about 50 mg of 5-OH-DMT.
47. The method of embodiment 37, comprising administering the tryptamine at the onset of the acute headache.
48. The method of embodiment 47, wherein the administration reduces the patient's pain associated with the acute headache.
49. A method of treating an acute headache and prophylactically preventing reoccurrence of acute headaches in a patient in need thereof, comprising

administering a first dose of tryptamine to the patient;

determining whether the first dose of tryptamine alleviated the patient's headache; and

if the first dose did not alleviate the patient's headache, administering a second dose of the tryptamine after administering the first dose,

wherein the tryptamine is 5-methoxy-N,N-dimethyltryptamine (5-MeO-DMT), 5-hydroxy-N,N-dimethyltryptamine (5-OH-DMT) or prodrugs or pharmaceutically acceptable salts thereof, and

wherein the patient experiences at least 2 migraine headaches a month during the 3 months prior to administration.

50. The method of embodiment 49, wherein the second dose of the tryptamine is administered 30 minutes after administration of the first dose of tryptamine.
51. The method of embodiment 49, wherein the second dose of the tryptamine is administered 1 hour after administration of the first dose of the tryptamine.
52. The method of embodiment 49, wherein the second dose of the tryptamine is administered 1 day after administration of the first dose of the tryptamine.
53. The method of embodiment 49, wherein the patient experiences at least 3 migraine headaches a month during the 3 months prior to administration.
54. The method of embodiment 49, wherein the patient experiences at least 4 migraine headaches a month during the 3 months prior to administration.
55. The method of embodiment 49, wherein the patient experiences at least 5 migraine headaches a month during the 3 months prior to administration.
56. The method of embodiment 49, wherein the patient exhibits a 30% reduction in a mean monthly number of migraines after administration of the tryptamine.
57. The method of embodiment 49, wherein the acute headache is an acute migraine.
58. The method of embodiment 49, wherein the acute headache is a cluster headache episode.
59. The method of embodiment 49, wherein the first and second dose of the tryptamine each comprise about 0.25 mg to about 50 mg of 5-MeO-DMT.
60. The method of embodiment 59, wherein the first and second dose of the tryptamine each comprise about 0.25 mg to about 36 mg of 5-MeO-DMT.
61. The method of embodiment 59, wherein the first and second dose of the tryptamine each comprise about 6 mg to about 30 mg of 5-MeO-DMT.
62. The method of embodiment 59, wherein the first and second dose of the tryptamine each comprise about 0.25 mg to about 3 mg of 5-MeO-DMT.
63. The method of embodiment 59, wherein the first and second dose of the tryptamine each comprise about 10 mg of 5-MeO-DMT.
64. The method of embodiment 49, wherein the first and second dose of the tryptamine each comprise about 0.25 mg to about 50 mg of 5-OH-DMT.
65. The method of embodiment 49, comprising administering the tryptamine at the onset of the acute headache.
66. The method of embodiment 65, wherein the administration reduces the patient's pain associated with the acute headache.

### EXAMPLES

### Example 1. Effects of 5-MeO-DMT and 5-OH-DMT in Headaches

5-MeO-DMT and/or 5-OH-DMT will be evaluated in one or more rodent models of headache, for example:
inhibition of rat trigeminovascular protein extravasation (neuroinflammatory response) upon electrical stimulation of the trigeminal nerve;
inhibition of rat dural-evoked trigeminovascular nociceptive transmission;
inhibition of ex vivo KCl-stimulated CGRP release from the mouse trigeminovascular system;
inhibition of capsaicin (IV)— or periarterial electrical stimulation-mediated dural artery vasodilation; or
inhibition of induction of c-fos in rat nociceptive neurons located in cervical, spinal substantia gelatinosa and brainstem nucleus caudalis.

Studies may include electrophysiological recordings from convergent wide-dynamic range neurons in trigeminal nucleus caudalis (TNC) following: a) infusion of glyceryl trinitrate (GTN), b) inflammatory soup applied to brain dura matter or c) 1% capsaicin applied to brain dura matter. Acute effects of vehicle, sumatriptan positive reference, or test article on spontaneous neuron activity and/or facilitation of mechanically-evoked neuron activity will be evaluated.

### Rodent Behavioral Study

A rodent behavioral study will investigate acute 5-MeO-DMT and/or 5-OH-DMT effects on periorbital hypersensitivity following infusion of inflammatory mediators. Male Sprague-Dawley rats will be cannulated to receive supradural infusions of an inflammatory soup containing histamine, bradykinin, serotonin and prostaglandin E2 under brief anesthesia once per day over 5 consecutive days. After the 5-day inflammatory soup sensitization period, rats demonstrating facial hypersensitivity will have a one-week wash-out period and then will be challenged with 0.1 mg/kg GTN, followed by acute vehicle, sumatriptan positive reference, or test article administration (e.g., 5-MeO-DMT and/or 5-OH-DMT), with at least a one-week washout between each test condition. The design is within subjects with the same animals receiving multiple treatments in a cross-over fashion (n=12/treatment). A blinded investigator will test periorbital mechanical sensitivity using von Frey filaments with reproducible calibrated buckling forces varying from 0.4-10 g utilizing the simplified up-and-down method before drug administration and 30 min, 1.5 h, and at 2.5 h after drug administration.

A positive response is characterized by several behavioral criteria: stroking the face with a forepaw, head withdrawal from the stimulus, and head shaking. Data will be expressed as the average±SEM and analyzed by two-way ANOVA followed by an appropriate post-hoc test to determine differences between treatment and control (Veh/GTN control group).

### In Vitro Testing

5-OH-DMT's 5-HT1B receptor agonism was investigated.

Methods: cAMP Secondary Messenger Pathway Assay Method.

A panel of cell lines stably expressing non-tagged GPCRs that signal through cAMP was developed. Hit Hunter® cAMP assays were used to monitor the activation of a GPCR via Gi and Gs secondary messenger signaling in a homogenous, non-imaging assay format using DiscoverX's Enzyme Fragment Complementation (EFC) with β-galactosidase (β-Gal) as the functional reporter. The enzyme is split into two complementary portions: EA for Enzyme Acceptor and ED for Enzyme Donor. ED is fused to cAMP and in the assay competes with cAMP generated by cells for binding to a cAMP-specific antibody. Active β-Gal is formed by complementation of exogenous EA to any unbound EDcAMP. Active enzyme can then convert a chemiluminescent substrate, generating an output signal detectable on a standard microplate reader.

cAMP Hunter cell lines were expanded from freezer stocks according to standard procedures. Human 5-HT1B receptor-mediated cAMP signaling was evaluated in stably transfected hamster-derived CHO-K1 cells. Cells were seeded in a total volume of 20 μL into white walled, 384-well microplates and incubated at 37° C. overnight. cAMP modulation was determined using the DiscoverX HitHunter cAMP XS+ assay.

For Gi agonist determination, cells were incubated with sample in the presence of 15 mM forskolin to induce response. Media was aspirated from cells and replaced with 10 μL HBSS/10 mM Hepes. Intermediate dilution of sample stocks was performed to generate 4× sample in assay buffer. 5 μL of 4× sample plus 5 μL of 4× forskolin was added to cells and incubated at 37° C. for 30 minutes. Final assay vehicle concentration was 1%. After compound incubation, assay signal was generated through incubation with 20 μL cAMP XS+ED/CL lysis cocktail for one hour followed by incubation with 20 μL cAMP XS+EA reagent for three hours at room temperature. Microplates were read following signal generation with a PerkinElmer Envision™ instrument for chemiluminescent signal detection.

Compound activity was analyzed using CBIS data analysis suite (ChemInnovation, CA). For Gi agonist mode assays, percentage activity is calculated using the following formula: % Activity=100%×(1−(mean RLU of test sample−mean RLU of MAX control)/(mean RLU of vehicle control−mean RLU of MAX control)). In these studies, the MAX RLU was generated by using serotonin (5-HT) as the reference agonist.

In a first experiment (US073-0021103), 5-OH-DMT was tested at 10 concentrations in duplicate, with a top concentration of 100 μM using 3-fold dilutions.

Result #1: In the first experiment, 5-OH-DMT exhibited full agonist activity with a potency of less than 0.005 mM at human 5-HT1B receptors (Table 1). The 5-HT reference agonist exhibited the anticipated full agonist activity with a potency of 0.0002 mM in this experiment.

TABLE 1
Results from Study #1

| | 5-HT1B | Curve Top | Maximal |
|---|---|---|---|
| Compound | EC50 (mM) | (% activation) | activation (%) |
| 5-OH-DMT | <0.00508 | Not | 103.64 |
| (hydrochloride) | | determined | |
| 5-HT (reference | 0.0002 | 100.22 | 101.21 |
| control) | | | |

The same human 5-HT1B receptor functional agonism assay as described above was used in a separate experiment. 5-OH-DMT was tested at 10 concentrations in duplicate, with a top concentration of 1 μM using 3-fold dilutions. This study demonstrates very potent full agonist activity, with the potential for an EC50≤1 nM.

Result #2: In the second experiment, 5-OH-DMT exhibits full agonist activity with a potency of 0.00009 mM at human 5-HT1B receptors (Table 2). The 5-HT reference agonist exhibited the anticipated full agonist activity with a potency of 0.00019 mM in this experiment. The effects of both test compounds in this study were highly consistent with the results of the first experiment, described above.

TABLE 2
Results from Study #2

| | 5-HT1B | Curve Top | Maximal |
|---|---|---|---|
| Compound | EC50 (mM) | (% activation) | activation (%) |
| 5-OH-DMT | 9.02298 × 10-5 | 100.41 | 96.661 |
| (hydrochloride) | (0.00009) | | |
| 5-HT (reference | 0.00019 | 107.54 | 105.94 |
| control) | | | |

Together with the available published data, these data demonstrated that 5-OH-DMT exhibits human 5-HT1B and 5-HT1F receptor agonist activity, consistent with known mechanisms of action of anti-migraine therapeutics. Available published data indicate that 5-MeO-DMT exhibits human 5-HT1B, 5-HT1D and 5-HT1F receptor agonist activity, consistent with known mechanisms of action of anti-migraine therapeutics.

### In Vivo Testing

In vivo testing includes doses within the anticipated non-psychoactive and psychoactive ranges (e.g., 5-MeO-DMT mouse head-twitch response elicited at doses of 10 mg/kg i.p. or s.c. and higher, Halberstadt et al, 2011, Dunlap et al., 2020; 5-OH-DMT mouse head-twitch response ED50=15 mg/kg s.c., Come and Pickering, 1967, may require confirmation). 5-OH-DMT may be evaluated in in vitro binding assays across a broad panel of targets to further characterize its pharmacological effects.

### Cardiovascular Safety Evaluation of 5-MeO-DMT

Cardiovascular safety evaluation of 5-MeO-DMT and/or 5-OH-DMT may include: confirming a lack of vasoconstrictive effects using, for example, ex vivo preparations of rabbit saphenous vein, dog coronary or carotid artery, or human proximal or distal coronary artery, meningeal or cerebral arteries.

### Example 2. Effects of 5-OH-DMT in a Stress Plus Sodium Nitroprusside-Induced Migraine Mice Model

To evaluate the prophylactic and acute/abortive treatment effects of single administration of 5-OH-DMT to female mice exposed to stress and sodium nitroprusside (SNP) as a model of stress-primed migraine pain.

### Developing Animal Model

C57BL/6j mice (7-8 weeks, female) were acclimated for 1 week prior to the experiment. Mice were kept in groups of 4-5 animals per cage in a housing room with temperature maintained at 21-23° C. and 40-70% relative humidity, on a 12/12 h light:dark cycle. Chow and water were available ad libitum except during testing. Animals were placed individually in cylindrical rodent restrainers and exposed to 2 hours of restraint stress for 3 consecutive days. von Frey testing of facial mechanical sensitivity was conducted 24 hours after the end of the final stress session (Day 1) to demonstrate that mice exposed to stress exhibited significantly elevated mechanical withdrawal scores indicative of hypersensitivity. On Day 10, von Frey testing was conducted again to confirm that mechanical withdrawal scores were similar across naïve and stress-exposed mice. Stress-exposed animals were randomized to treatment groups based on body weight and facial mechanical withdrawal score. Then, each animal was administered a 1st intraperitoneal (i.p.) injection of 5-OH-DMT (dissolved in 0.9% sterile saline and administered at 3, 10 or 30 mg/kg in a volume of 10 ml/kg) or vehicle.

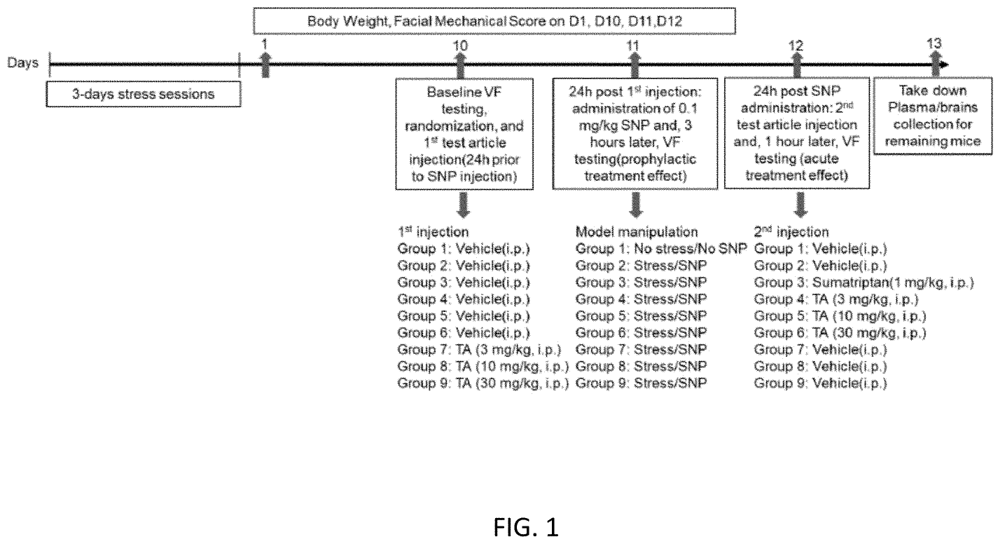

On Day 11, at 24 h following the 1^{st} injection, vehicle or 0.1 mg/kg sodium nitroprusside (SNP) was given i.p. and facial mechanical withdrawal scores were assessed 3 hours later to confirm establishment of a stress+SNP-induced hypersensitive state in Day 10 vehicle-treated animals (Groups 2-6) or, in Day 10 test article-treated rats (Groups 7-9), assess the prophylactic effect of compound administration. On Day 12, at 23 hours after vehicle or SNP injection, each animal was administered a 2nd i.p. injection of vehicle or 5-OH-DMT or sumatriptan (acute treatment positive control animals received vehicle on Day 10). One hour later, facial withdrawal scores were assessed to evaluate acute effects of treatments (Groups 2-6) or durability of prophylactic effects (Groups 7-9). The overall study design is summarized in FIG. 1.

### Measurements

Mice were allowed to acclimate to the testing cage for 60 minutes each day before the test. To assess pain-like behaviors induced by noxious mechanical stimulation applied on the vibrissal pad (orofacial area), the mouse was placed in a small cage with a metal mesh ceiling and a wood bottom (10 cm×5 cm×5 cm). The semiparametric scoring system used to evaluate the response of the animal to the mechanical stimulation was described in detail by Vos et al. (1994). Mechanical stimulation delivered by a von Frey hair with the bending force of 0.07 g was applied bilaterally to the vibrissal pad. The scoring system for evoked responses was the following: 0=no response, 0.5=subthreshold response, 1=threshold response (weak withdrawal of the stimulated site), 1.5=threshold response (quick withdrawal of the stimulated site), 2=suprathreshold response (a strong withdrawal that involves more than the stimulated site), 3=suprathreshold response with whole body turning around. Mechanical stimulation was applied to each mouse 8 times (4 times on the left side and 4 times on the right side) with an interval of at least 5 minutes between each stimulation. The summated score of these 8 trials was counted as the animal's response score. The testing cage was cleaned with 75% ethanol to remove odor, urine and feces, prior to testing each animal.

### Sample Collection

Terminal brain and plasma samples were collected from a subset of n=4 animals from 5-OH-DMT groups on Day 12 as soon after behavioral testing as possible (exact time post dose was recorded). Animals were euthanized by carbon dioxide (CO₂) inhalation. Blood samples were collected through cardiac puncture and collected into tubes pre-coated with EDTA-K2 for plasma preparation. Plasma (˜200 uL/animal) was obtained by centrifugation at 4000 rpm for 10 minutes at 4° C. and then stored at −80° C. for bioanalysis. The mice were perfused with normal saline, and brains were collected and stored at −80° C. for bioanalysis. The remaining study animals were euthanized on Day 13 by CO₂ inhalation.

### Bioanalysis

5-OH-DMT concentrations in biological samples were analyzed by liquid chromatography-mass spectrometry (LC/MS/MS). A calibration curve was constructed in blank plasma or brain homogenate. Non-compartmental analysis: PK parameters including T½, AUC, Cmax, Tmax, and MRT etc. were calculated by WinNonlin software (version 8.3). BLOQ means below lower limit of quantification (3 ng/mL). Samples with BLOQ were set to 1.5 ng/ml (half of the lower limit of detection). Chromatographic separation was achieved on the Waters ACQUITY UPLC BEH C18 column 2.1*50 mm 1.7 um. The column temperature was maintained at RT. The flow rate was maintained at 0.6 mL/min. Linearity, LLOQ, QC and accuracy were determined during the process.

Plasma: An aliquot of 20 μL plasma was spiked into a 1.5 mL tube, and 80 μL of acetonitrile containing internal standard was added for protein precipitation. The mixture was vortexed and centrifuged at 14000 rpm for 15 min. After transfer of 80 μL of supernatant and mixing with 320 μL of water, the final solution was injected for LC-MS/MS analysis.

Brain: Brain samples were homogenized with ice-cold phosphate buffer saline (pH 7.4) at a ratio of 4 (buffer):1(cartilage tissue) (v/w). An aliquot of 20 μL homogenate was spiked into 1.5 mL tube, and 80 μL of acetonitrile containing internal standard was added for protein precipitation. The mixture was vortexed and centrifuged at 14000 rpm for 5 min. After transfer of 80 μL supernatant into a 96 well plate and the addition of 240 μL water, the mixture was vortexed and injected for LC-MS/MS analysis.

### Results

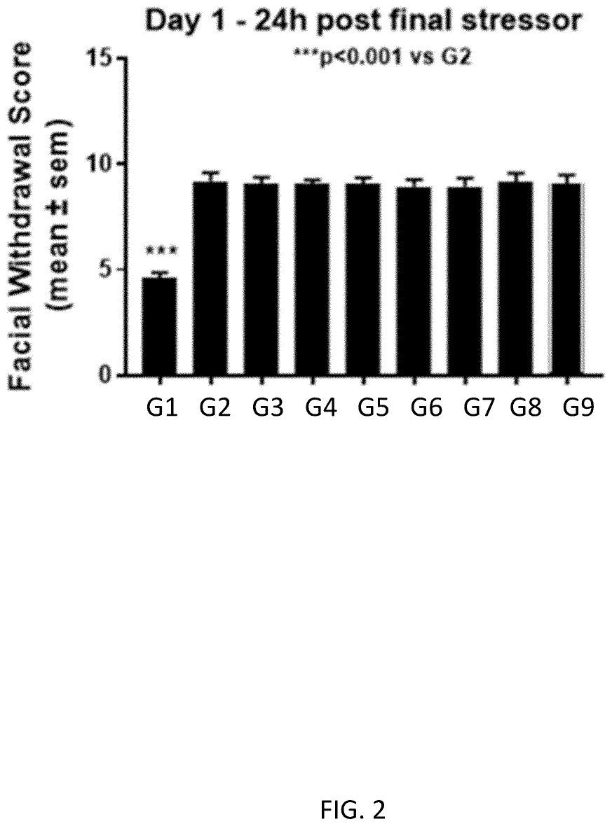

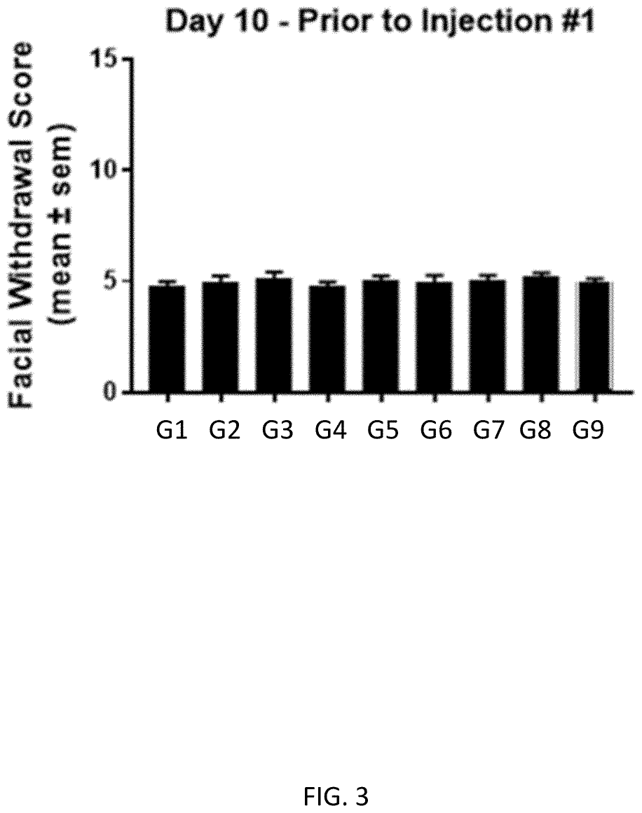

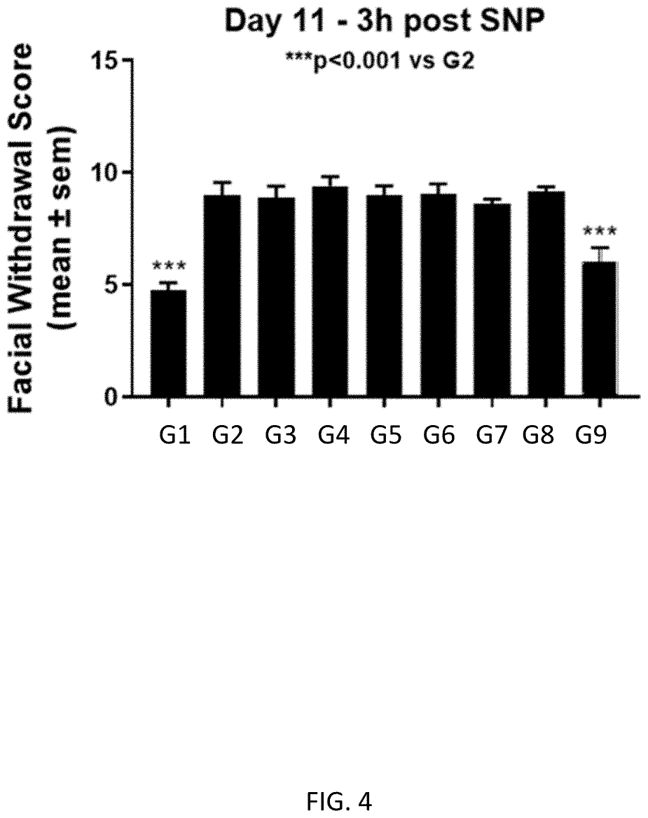

Exposure to 3 consecutive days of 2 h restraint stress and ending 24 h prior to testing induced a hypersensitive state in the animals exposed to stress (Groups 2-9, G2-G9) compared to those that were not exposed to stress (Group 1, G1) (FIG. 2). The mice that had been exposed to repeated stress (G2-G9) no longer exhibited facial hypersensitivity on Day 10 after stress, compared to animals that were not exposed to stress (G1) (FIG. 3). After behavioral testing on Day 10, animals were administered injection #1 (vehicle to G1-G6, or a dose of 5-OH-DMT to G7-G9) 24 h prior to SNP, to evaluate if single administration of 5-OH-DMT exhibited a prophylactic effect to reduce stress+SNP-induced hypersensitivity. On Day 11, animals previously exposed to stress were administered SNP (G2-G9), while animals without stress exposure were administered vehicle (G1). Three hours after SNP or vehicle injection, facial hypersensitivity was assessed. Confirming that the experimental manipulation of stress+SNP induced a hypersensitive state, there was a significant difference in facial withdrawal scores between the vehicle-treated stress+SNP (G2) and no stress+SNP (G1) groups (FIG. 4). In animals exposed to stress+SNP, prophylactic treatment with 30 mg/kg 5-OH-DMT (G9) significantly reduced facial hypersensitivity compared to vehicle treatment (G2) (FIG. 4).

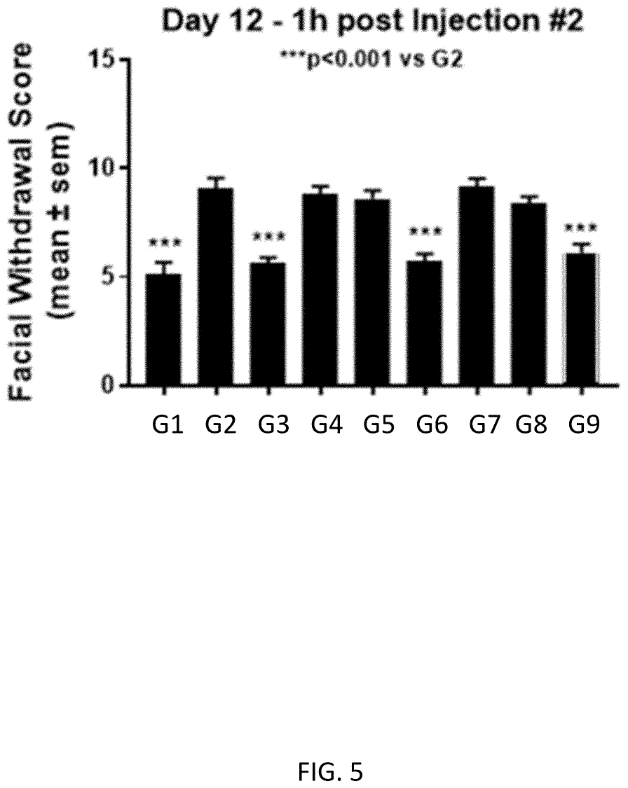

On Day 12, 24 h after SNP administration, animals were given injection #2 (vehicle to G1-G2 and G7-G9, sumatriptan positive control to G3, or a dose of 5-OH-DMT to G4-G6), to evaluate if single administration of 5-OH-DMT exhibited an acute/abortive treatment effect to reduce stress+SNP-induced hypersensitivity. One hour after injection #2, facial hypersensitivity was assessed. Confirming the persistence of the stress+SNP-induced hypersensitive state, there was a significant difference in facial withdrawal scores between the vehicle-treated stress+SNP (G2) and no stress+SNP (G1) groups on Day 12 (FIG. 5). In animals exposed to stress+SNP, acute treatment with sumatriptan (G3) or 30 mg/kg 5-OH-DMT (G6) significantly reduced facial hypersensitivity compared to vehicle treatment (G2) (FIG. 5). Confirming the durability of the prophylactic effect, animals given 30 mg/kg 5-OH-DMT (G9) on Day 10 still showed significantly reduced facial hypersensitivity compared to vehicle treatment (G2) on Day 12 (FIG. 5).

### CONCLUSIONS

Single administration of 5-OH-DMT at 30 mg/kg exhibited significant reductions in facial hypersensitivity in a model of stress-primed migraine pain. The significant anti-hyperalgesic effects of 30 mg/kg 5-OH-DMT were observed following single prophylactic or acute/abortive treatment to female mice exposed to stress+SNP. The prophylactic effect of single administration of 5-OH-DMT was durable over the 2-day assessment period. These result support the utility of single administration of 5-OH-DMT in the prophylactic or acute/abortive treatment of migraine and/or orofacial pain.

### INCORPORATION BY REFERENCE

All references, articles, publications, patents, patent publications, and patent applications cited herein are incorporated by reference in their entireties for all purposes. However, mention of any reference, article, publication, patent, patent publication, and patent application cited herein is not, and should not be taken as, an acknowledgment or any form of suggestion that they constitute valid prior art or form part of the common general knowledge in any country in the world.

What is claimed is:

1. A method of treating facial pain or headache in a patient in need thereof comprising administering a therapeutically effective amount of a tryptamine comprising 5-methoxy-N,N-dimethyltryptamine (5-MeO-DMT), 5-hydroxy-N,N-dimethyltryptamine (5-OH-DMT) or prodrugs or pharmaceutically acceptable salts thereof.

2. The method of claim 1, wherein the facial pain is selected from the group consisting of temporomandibular disorder (TMD) associated pain, neuralgias, trigeminal neuraligia, glossopharyngeal neuralgia, sphenopalatine ganglion neuralgia, postherpetic neuralgia, Sluder's neuralgia, mental nerve neuralgia, post-injury associated pain, burning mouth syndrome associated pain, persistent idiopathic associated pain, atypical odontalgia associated pain, mucosal ulceration associated pain, ulceration associated pain, traumatic ulceration associated pain, immunologic ulceration associated pain, infective ulceration associated pain, erosive ulceration associated pain, vesiculobullous associated pain, psychosomatic related pain, sinonasal related pain, rhinosinusitis associated pain, salivary gland disease associated pain, cardiac toothache associated pain, eagle syndrome associated pain, myofascial related pain, muscle spasm associated pain, dental related pain, craniofacial pain syndrome, temporal tendonitis associated pain, ernest syndrome associated pain, hamular bursitis associated pain, occipital neuritis associated pain, cervicalgia associated pain, fibromyalgia associated pain, trigeminal autonomic cephalalgias associated pain, cranial neuropathies associated pain, and pain associated with headache.

3. The method of claim 2, wherein the facial pain is a trigeminal neuraligia.

4. The method of claim 1, wherein the headache is a migraine, classic migraine, migraine disorder, migraine without aura, migraine with aura, chronic migraine, acute migraine, episodic migraine, vestibular migraine, menstrual migraine, muscular character of migraine, headache, acute headache, chronic headache, tension type headache, medication overuse headache, cluster headache, post traumatic headache, postdural puncture headache, chronic cluster headache, episodic cluster headache, infrequent episodic tension-type headache, frequent episodic tension-type headache, or chronic tension-type headache.

5. The method of claim 4, wherein the headache is an acute headache.

6. The method of claim 4, wherein the headache is an acute migraine.

7. The method of claim 4, wherein the headache is a chronic headache or a chronic migraine.

8. The method of claim 4, wherein the headache is a cluster headache episode.

9. The method of claim 1, wherein the method comprises administering a therapeutically effective amount of 5-MeO-DMT or a prodrug or a pharmaceutically acceptable salt thereof to the patient.

10. The method of claim 9, wherein the method comprises administering between about 0.25 mg to about 50 mg of 5-MeO-DMT.

11. The method of claim 9, wherein the method comprises administering between about 0.25 mg to about 36 mg of 5-MeO-DMT.

12. The method of claim 9, wherein the method comprises administering between about 6 mg to about 30 mg of 5-MeO-DMT.

13. The method of claim 9, wherein the method comprises administering between about 0.25 mg to about 3 mg of 5-MeO-DMT.

14. The method of claim 9, wherein the method comprises administering about 10 mg of 5-MeO-DMT.

15. The method of claim 1, wherein the method comprises administering a therapeutically effective amount of 5-OH-DMT or a prodrug or a pharmaceutically acceptable salt thereof to the patient.

16. The method of claim 15, wherein the method comprises administering between about 0.25 mg to about 50 mg of 5-OH-DMT.

17. The method of claim 1, wherein the method comprises administering the tryptamine by insufflation.

18. The method of claim 1, wherein the method comprises administering the tryptamine intranasally.

19. The method of claim 1, wherein the method comprises administering the tryptamine orally.

20. The method of claim 1, wherein the method comprises administering the tryptamine subcutaneously.

21. The method of claim 1, wherein the method comprises administering the tryptamine sublingually.

22. The method of claim 1, wherein the method comprises administering the tryptamine buccally.

23. The method of claim 1, wherein the method comprises administering the tryptamine by inhalation.

24. The method of claim 1, wherein the method comprises administering a single dose of the tryptamine to the patient.

25. The method of claim 24, wherein the method further comprises administering a second dose of tryptamine to the patient about 30 minutes after a first administration if the first dose is not therapeutically effective.

26. The method of claim 1, wherein the method comprises administering the tryptamine at the onset of the facial pain or headache.

27. The method of claim 1, wherein administering the tryptamine reduces the visual analog scale (VAS) score by at least about 1 point compared to the VAS score of the patient prior to treatment.

28. A method of treating and prophylactically preventing reoccurrence of acute headaches in a patient in need thereof, the method comprising administering a therapeutically effective amount of a tryptamine comprising 5-methoxy-N,N-dimethyltryptamine (5-MeO-DMT), 5-hydroxy-N,N-dimethyltryptamine (5-OH-DMT) or prodrugs or pharmaceutically acceptable salts thereof to the patient, wherein the patient experiences at least 2 migraine headaches a month during the 3 months prior to administration.

29. The method of claim 28, comprising administering at least two doses of the tryptamine to the patient in need thereof.

30. The method of claim 28, comprising administering at least three doses of the tryptamine to the patient in need thereof.

31. The method of claim 29, wherein the at least two doses of the tryptamine are administered 30 minutes (min) apart.

32. The method of claim 30, wherein the at least three doses of the tryptamine are administered 30 min apart.

33. The method of claim 28, wherein the patient experiences at least 3 headaches a month during the 3 months prior to administration.

34. The method of claim 28, wherein the patient experiences at least 4 headaches a month during the 3 months prior to administration.

35. The method of claim 28, wherein the patient experiences at least 5 headaches a month during the 3 months prior to administration.

36. The method of claim 28, wherein the patient exhibits a 30% reduction in a mean monthly number of migraines after administration of the tryptamine.

37. The method of claim 28, wherein the migraine headache is an acute migraine.

38. The method of claim 28, wherein the migraine headache is a cluster headache episode.

39. The method of claim 28, comprising administering a therapeutically effective amount of 5-MeO-DMT or a prodrug or a pharmaceutically acceptable salt thereof to the patient.

40. The method of claim 39, comprising administering between about 0.25 mg to about 50 mg of 5-MeO-DMT.

41. The method of claim 39, comprising administering between about 0.25 mg to about 36 mg of 5-MeO-DMT.

42. The method of claim 39, comprising administering between about 6 mg to about 30 mg of 5-MeO-DMT.

43. The method of claim 39, comprising administering between about 0.25 mg to about 3 mg of 5-MeO-DMT.

44. The method of claim 39, comprising administering about 10 mg of 5-MeO-DMT.

45. The method of claim 28, comprising administering a therapeutically effective amount of 5-OH-DMT or a prodrug or a pharmaceutically acceptable salt thereof to the patient.

46. The method of claim 45, comprising administering between about 0.25 mg to about 50 mg of 5-OH-DMT.

47. The method of claim 37, comprising administering the tryptamine at the onset of the acute headache.

48. The method of claim 47, wherein the administration reduces the patient's pain associated with the acute headache.

49. A method of treating an acute headache and prophylactically preventing reoccurrence of acute headaches in a patient in need thereof, comprising
administering a first dose of tryptamine to the patient;
determining whether the first dose of tryptamine alleviated the patient's headache; and
if the first dose did not alleviate the patient's headache, administering a second dose of the tryptamine after administering the first dose,
wherein the tryptamine is 5-methoxy-N,N-dimethyltryptamine (5-MeO-DMT), 5-hydroxy-N,N-dimethyltryptamine (5-OH-DMT) or prodrugs or pharmaceutically acceptable salts thereof, and
wherein the patient experiences at least 2 migraine headaches a month during the 3 months prior to administration.

50. The method of claim 49, wherein the second dose of the tryptamine is administered 30 minutes after administration of the first dose of tryptamine.

51. The method of claim 49, wherein the second dose of the tryptamine is administered 1 hour after administration of the first dose of the tryptamine.

52. The method of claim 49, wherein the second dose of the tryptamine is administered 1 day after administration of the first dose of the tryptamine.

53. The method of claim 49, wherein the patient experiences at least 3 migraine headaches a month during the 3 months prior to administration.

54. The method of claim 49, wherein the patient experiences at least 4 migraine headaches a month during the 3 months prior to administration.

55. The method of claim 49, wherein the patient experiences at least 5 migraine headaches a month during the 3 months prior to administration.

56. The method of claim 49, wherein the patient exhibits a 30% reduction in a mean monthly number of migraines after administration of the tryptamine.

57. The method of claim 49, wherein the acute headache is an acute migraine.

58. The method of claim 49, wherein the acute headache is a cluster headache episode.

59. The method of claim 49, wherein the first and second dose of the tryptamine each comprise about 0.25 mg to about 50 mg of 5-MeO-DMT.

60. The method of claim 59, wherein the first and second dose of the tryptamine each comprise about 0.25 mg to about 36 mg of 5-MeO-DMT.

61. The method of claim 59, wherein the first and second dose of the tryptamine each comprise about 6 mg to about 30 mg of 5-MeO-DMT.

62. The method of claim 59, wherein the first and second dose of the tryptamine each comprise about 0.25 mg to about 3 mg of 5-MeO-DMT.

63. The method of claim 59, wherein the first and second dose of the tryptamine each comprise about 10 mg of 5-MeO-DMT.

64. The method of claim 49, wherein the first and second dose of the tryptamine each comprise about 0.25 mg to about 50 mg of 5-OH-DMT.

65. The method of claim 49, comprising administering the tryptamine at the onset of the acute headache.

66. The method of claim 65, wherein the administration reduces the patient's pain associated with the acute headache.
